(19) United States
(12) Patent Application Publication
(10) Pub. No.: US 2008/0149612 A1
(43) Pub. Date: Jun. 26, 2008

(54) Heating support for patients

(75) Inventor: Axel Stihler, Stuttgart (DE)

Correspondence Address: KOHLER SCHMID MOEBUS, RUPPMANNSTRASSE 27, D-70565 STUTTGART, omitted

(73) Assignee: Stihler Electronic medizinische Geraete Produktions- und Vertriebs-GmbH, Stuttgart (DE)

(21) Appl. No.: 12/000,796

(22) Filed: Dec. 18, 2007

(30) Foreign Application Priority Data
Dec. 22, 2006 (DE) 10 2006 062 233.2

(51) Int. Cl. H05B 1/00; A61F 7/08

(52) U.S. Cl. 219/212; 607/112

(57) ABSTRACT

A heating support (1) for a patient comprises a heating mat or a heating blanket (2) of a heat-insulating material, at least one heating element (3) that is integrated into the heating mat or heating blanket (2), and at least one temperature sensor (4). The heating support (1) is divided into a patient support area (7) suited for diagnostic imaging applications, and a temperature measurement area (9).

This application claims Paris Convention priority of DE 10 2006 062 233.2 filed Dec. 22, 2006 the complete disclosure of which is hereby incorporated by reference.

### BACKGROUND OF THE INVENTION

The present invention relates to an arrangement for controlling a heating support for a patient, which comprises a heating mat or heating blanket made of a heat-insulating material, at least one heating element that is integrated into the heating mat or heating blanket, and at least one temperature sensor, wherein the heating support is divided into a patient support area that is suitable for diagnostic imaging applications, and a temperature measurement area.

A heating support for patients of this type is disclosed e.g. in DE 10 2005 019 868 A1.

Heating supports for patients are used for medical examinations, operations and/or monitoring of patients who are in intensive care. Correct adjustment of the temperature of the heating support by the physician or the nursing staff is important. Towards this end, corresponding control means and temperature sensors that are connected to the control means are provided for controlling and adjusting the heating current that flows through the heating element.

It is thereby problematic that the temperature sensors and some heating elements that are used are not suited for diagnostic imaging applications, since they are not produced from a material that is transparent in accordance with these applications.

For this reason, the above-mentioned published document proposes not only to use a temperature sensor that is connected to the heating device but also to evaluate at least one further temperature signal related to a body feature of the patient for controlling the heating power.

Measurement of the temperature on the skin surface of the patient is not very accurate and therefore rather unsuitable for controlling the heating device.

Temperature measurement on the patient is more demanding or interferes with the medical application.

It is therefore the underlying purpose of the invention to improve the conventional arrangement for controlling the heating support in such a fashion that handling is facilitated and the application of diagnostic imaging methods is not disturbed.

### SUMMARY OF THE INVENTION

This object is achieved by an arrangement in accordance with the independent claim. Measurement on the patient is simulated in the temperature measurement area. For this reason, the remaining area of the heating support for a patient need not contain any temperature sensors. By simulating the patient, the heating support for a patient can be adjusted to the patient without the patient being supported on the sensor field of the remaining support for a patient.

The temperature measurement area is advantageously completely integrated into the heating support for a patient.

For application of diagnostic medical imaging methods, the at least one heating element is formed by a carbon fiber.

Further advantageous further developments of the invention are described in the claims.

The drawing schematically shows one preferred embodiment of the invention, which is described below with reference to the figures of the drawing.

### BRIEF DESCRIPTION OF THE DRAWING

FIG. 1 shows a side view of a heating support;

FIG. 2 shows a top view of the heating support.

### DESCRIPTION OF THE PREFERRED EMBODIMENT

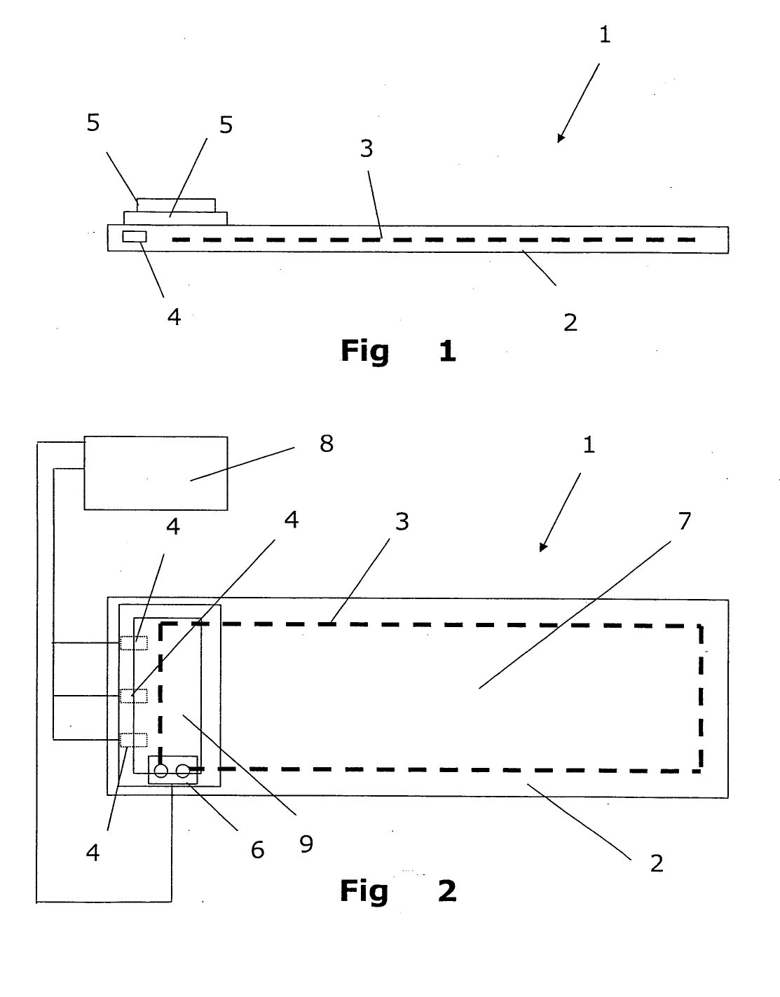

FIG. 1 shows that a heating support 1 comprises a heating blanket or a heating mat 2 into which a heating element 3 is integrated. The heating blanket or heating mat 2 is produced from a heat-insulating material. This may e.g. be a material that is distributed under the trademark Thinsulate. Thinsulate™ insulation is a non-woven material that is used as heat insulation in textiles and bedding or also as acoustical insulation material in the automotive industry. The heating element 3 is formed by a conductor, such that the heating support 1 can be heated by a heating current. The heating element 3 may be designed as a carbon fiber which is transparent to X-rays and also has a low magnetic reduction, such that the heating element 3 can be used without any problem when the heating support 1 is used for diagnostic imaging applications. The heating support 1 is divided into an area on which the patient to be examined is supported during the examination, and one area that bears the electric connections as well as the temperature sensors 4 for controlling the heating support 1. NTC sensors are particularly suited as temperature sensors. NTC sensors are temperature-independent semi-conductor resistors whose conductivity is better at higher temperatures than at lower temperatures. For this reason, they are particularly suited for application in heating supports. One area is designed transparent for diagnostic imaging applications, while the other area is not transparent due to the temperature sensors and the electric connections for diagnostic imaging applications. Several layers of the heat-insulating material 5 are disposed above the temperature sensors 4. The number of layers of this material is adjusted in such a fashion that it is not necessary to measure the temperature in that area where the patient is supported. Several layers of the heat-insulating material simulate a patient lying above the temperature sensors. Temperature measurement directly on the patient is also not necessary.

As is shown in FIG. 2, the heating element 3 is guided parallel to the edge of the heating support 1, wherein the electric connection 6 is formed on one side of the heating support 1. This produces a large transparent support area 7 for diagnostic imaging applications. For defined control of the heating support 1, the temperature sensors 4 and the electric connection 6 are connected to a control unit 8 in order to control the heating current through the heating element 3 in such a fashion that a temperature of usually more than 36° C. is obtained for heating the patient. Measurement is thereby performed in that area 9 where the patient is not supported. The patient is simulated in this area by the layers of heat-insulating material. The control means 8 may comprise a data storage in which the characteristics for heating the patient are stored. The characteristics may be based on previous measurements, experience and a series of tests, which show which combination between number of layers of heat-insulating material and heating current can be associated with a defined heating of the heating support 1. Several layers of heat-insulating material form a standard patient as it were.

I claim:

1. A device for controlling a heating support for a patient, the device comprising:
a heating mat or a heating blanket having a heat-insulating material;
at least one heating element integrated into said heating mat or heating blanket;
means for defining, within said heating support, a patient support area suited for diagnostic imaging applications and a temperature measurement area;
at least one temperature sensor disposed at said temperature measurement area; and
several layers of heat-insulating material structured and dimensioned to simulate the patient and disposed in said temperature measurement area.

2. The device of claim 1, wherein three temperature sensors are provided in said temperature measurement area above which several layers of said heat-insulating material are disposed.

3. The device of claim 1, wherein said the temperature sensor and said layers of heat-insulating material are integrated into said heating mat or heating blanket.

4. The device of claim 1, wherein said heating element is formed by a carbon fiber.
